(19) United States
(12) Patent Application Publication
(10) Pub. No.: US 2010/0075997 A1
(43) Pub. Date: Mar. 25, 2010

(54) USE OF PKC INHIBITORS IN TRANSPLANTATION

(76) Inventors: Alexander Korn, Allschwill (CH); Philip Lake, Morris Plains, NJ (US); Axel Maibucher, Allschwill (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/517,207

(22) PCT Filed: Dec. 5, 2007
(86) PCT No.: PCT/US07/86463

§ 371 Date: Jun. 2, 2009

Related U.S. Application Data
(60) Provisional application No. 60/868,991, filed on Dec. 7, 2006.

(51) Int. Cl. A61K 31/517; A61K 31/404; A61K 31/44; A61P 3/10; A61P 37/06

(52) U.S. Cl. 514/266.2; 514/414; 514/299

(57) ABSTRACT

The present invention pertains to the use of a PKC inhibitor in the treatment of insulin-producing cell and tissue rejection, such as islet transplantation rejection or rejection of transdifferentiated insulin-producing hepatocytes.

The present invention relates to the use of a PKC inhibitor in the treatment or prevention of rejection of insulin-producing cells or tissues such as islet transplantation rejection, in particular pancreatic islet cell transplantation, or preventing or delaying of rejection of insulin-producing tissues such as transdifferentiated hepatocytes, for example for the treatment of diabetes.

Transplantation of an entire pancreas is a major surgery, requiring general anesthetic, long stay in hospital and carries associated surgical and anesthetic risks. Transplantation of islet cells is a less invasive procedure, requiring only a local anesthetic. The islet cells may be isolated from the pancreas of a donor and then injected through a thin needle into the recipient's umbilical vein in the abdomen or through a tube inserted into a vein to the liver. Once transplanted, the new islet cells make and release insulin.

In spite of numerous treatment options for treating or preventing these graft rejections, there is still the need to expand the armamentarium of available immunosuppressive drugs available to increase the long-term success rate of islet transplantation, i.e. to treat, prevent or delay islet transplant rejection.

The present invention provides the use of a PKC inhibitor, in particular an indolylmaleimide derivative, in preventing, treating or delaying islet transplantation rejection, in particular in case of diabetes or chronic pancreatitis, wherein the indolylmaleimide derivative is a compound of formula (I)

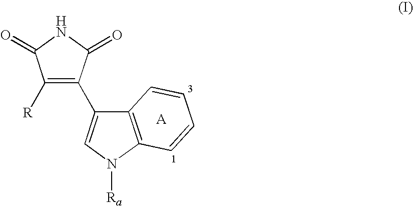

wherein
Rₐ is H; C₁₋₄alkyl; or C₁₋₄alkyl substituted by OH, NH₂, NHC₁₋₄alkyl or N(di-C₁₋₄alkyl)₂; and
R is a radical of formula (a) or (b)

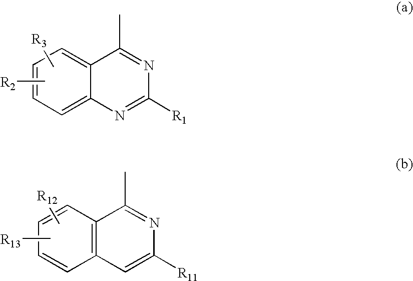

wherein
each of R₁ and R₁₁ is a heterocyclic residue; NR₄R₅ wherein R₄ and R₅ form together with the nitrogen atom to which they are bound a heterocyclic residue;
each of R₂, R₃, R₁₂ and R₁₃, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN; and
ring A is optionally substituted,
or a pharmaceutically acceptable salt thereof.

In formula (I), any alkyl or alkyl moiety in e.g. alkoxy may be linear or branched. Halogen may be F, Cl, Br or I, preferably F or Cl. Any aryl may be phenyl or naphthyl, preferably phenyl.

By heterocyclic residue as R₁, or R₁₁, or formed by NR₄R₅, is meant a three to eight, preferably five to eight, membered saturated, unsaturated or aromatic heterocyclic ring comprising 1 or 2 heteroatoms, preferably selected from N, O and S, and optionally substituted.

Suitable examples of heterocyclic residue as R₁, R₁₁ or formed by NR₄R₅ include e.g. pyridyl, e.g. 3- or 4-pyridyl, piperidyl, e.g. piperidin-1-yl, 3- or 4-piperidyl, homopiperidyl, piperazinyl, e.g. 1-piperazinyl, homopiperazinyl, morpholin-4-yl, imidazolyl, imidazolidinyl, pyrrolyl or pyrrolidinyl, optionally substituted, e.g. mono- or polysubstituted. When the heterocyclic residue is substituted, this may be on one or more ring carbon atoms and/or on a ring nitrogen atom when present. Examples of a substituent on a ring carbon atom include e.g. C₁₋₄alkyl e.g. CH₃;

C₃₋₆cycloalkyl e.g. cyclopropyl, optionally further substituted by C₁₋₄alkyl;

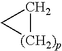

wherein p is 1,2 or 3, preferably 1; CF₃; halogen; OH; NH₂; —CH₂—NH₂; —CH₂—OH; piperidin-1-yl; or pyrrolidinyl. Examples of a substituent on a ring nitrogen atom are e.g. C₁₋₆alkyl; acyl, e.g. R′ₓ—CO wherein R′ₓ is H, C₁₋₆alkyl or phenyl optionally substituted by C₁₋₄alkyl, C₁₋₄alkoxy or amino, e.g formyl; C₃₋₆cycloalkyl; C₃₋₆cycloalkyl-C₁₋₄alkyl; phenyl; phenyl-C₁₋₄alkyl e.g. benzyl; a heterocyclic residue, e.g. as disclosed above, e.g. an aromatic heterocyclic residue comprising 1 or 2 nitrogen atoms; or a residue of formula α

—R₂₁—Y′ (

[symbol]

wherein R₂₁ is C₁₋₄alkylene or C₂₋₄alkylene interrupted by O and Y′ is OH, NH₂, NH(C₁₋₄alkyl) or N(C₁₋₄alkyl)₂.

In formula (I) C₂₋₄alkylene interrupted by O may be e.g. —CH₂—CH₂—O—CH₂—CH₂—.

In formula (I), when the substituent on a cyclic nitrogen is a heterocyclic residue, it may be a five or six membered saturated, unsaturated or aromatic heterocyclic ring comprising 1 or 2 heteroatoms, preferably selected from N, O and S. Examples include e.g. 3- or 4-pyridyl, piperidyl, e.g. piperidin-1-yl, 3- or 4-piperidyl, homopiperidyl, piperazinyl, homopiperazinyl, pyrimidinyl, morpholin-4-yl, imidazolyl, imidazolidinyl, pyrrolyl or pyrrolidinyl,

In formula (I), when Rₐ is substituted C₁₋₄alkyl, the substituent is preferably on the terminal carbon atom.

When ring A is substituted, it may be mono- or polysubstituted, preferably monosubstituted, the substituent(s) being selected from the group consisting of e.g. halogen, OH, C₁₋₄alkoxy, e.g. OCH₃, C₁₋₄alkyl, e.g. CH₃, NO₂, CF₃, NH₂, NHC₁₋₄alkyl, N(di-C₁₋₄alky1)₂ and CN. For example, ring A may be a residue of formula

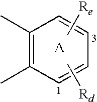

wherein
R_{d} is H; C₁₋₄alkyl; or halogen; and
Rₑ is OH; NO₂; NH₂; NHC₁₋₄alkyl; or N(di-C₁₋₄alkyl)₂.

Preferably R_{d} is in position 1; preferably Rₑ is in position 3.

When R_{c} has a CH₂ replaced by CRₓR_{y}, it is preferably the CH₂ bearing Y.

Examples of heterocyclic residue as R₁, R₁₁, or formed by NR₄R₅ include e.g. a residue of formula (γ)

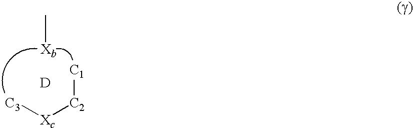

wherein
the ring D is a 5, 6 or 7 membered saturated, unsaturated or aromatic ring;
X_{b} is —N—, —C= or —CH—;
X_{c} is —N=, —NR_{f}—, —CR′_{f}= or —CHR′_{f}— wherein R_{f} is a substituent as indicated above for a ring nitrogen atom, and R′_{f} is a substituent as indicated above for a ring carbon atom;
the bond between C₁ and C₂ is either saturated or unsaturated;
each of C₁ and C₂, independently, is a carbon atom which is optionally substituted by one or two substituents selected among those indicated above for a ring carbon atom; and
the line between C₃ and X_{b} and between C₁ and X_{b}, respectively, represents the number of carbon atoms as required to obtain a 5, 6 or 7 membered ring D.

A preferred residue of formula (γ) is one wherein the ring D forms a 1,4-piperazinyl ring optionally C- and/or N-substituted as indicated.

Representative examples of a residue of formula (γ) are e.g. 3- or 4-pyridyl; piperidin-1-yl; 1-N-(C₁₋₄alkyl)- or -(ω-hydroxy-C₁₋₄alkyl)-3-piperidyl; morpholin-4-yl; imidazolyl; pyrrolidinyl; 1-piperazinyl; 2-C₁₋₄alkyl- or -C₃₋₆cycloalkyl-1-piperazinyl; 3-C₁₋₄alkyl- or -C₃₋₆cycloalkyl-1-piperazinyl; 2,2- or 3,5- or 2,5- or 2,6-di(C₁₋₄alkyl)-1-piperazinyl; 3,4,5-tri-(C₁₋₄alkyl)-1-piperazinyl; 4-N-(C₁₋₄alkyl)- or -(ω-hydroxy-C₁₋₄alkyl)- or -(ω-dimethylamino-C₁₋₄alkyl)-1-piperazinyl; 4-N-pyridin-4-yl-1-piperazinyl; 4-N-phenyl- or C₃₋₆cycloalkyl-1-piperazinyl; 4-N-(C₁₋₄alkyl)-or -(ω)-hydroxy-C₁₋₄alkyl)-3-C₁₋₄alkyl- or -3,3-di(C₁₋₄alkyl)-1-piperazinyl; 4-N-(1-C₁₋₄alkyl-C₃₋₆cycloalkyl)-1-piperazinyl; 4-N-formyl-1-piperazinyl; 4-N-pyrimidin-2-yl-1-piperazinyl; 4,7-diaza-spiro[2.5]oct-7-yl or 4-N-C₁₋₄alkyl-1-homopiperazinyl.

The compounds of formula (I) may exist in free form or in salt form, e.g. addition salts with e.g. organic or inorganic acids, for example, hydrochloric acid, acetic acid, when R₁ or R₁₁ and/or R₂, R₃, R₁₂ or R₁₃ comprises an optionally substituted amino group or a heterocyclic residue which can form acid addition salts.

It will be appreciated that the compounds of formula (I) may exist in the form of optical isomers, racemates or diastereoisomers. For example, a ring carbon atom bearing a substituent in the heterocyclic residue as R₁, R₁₁ or formed by NR₄R₅ is asymmetric and may have the D- or L-configuration. It is to be understood that the present invention embraces all enantiomers and their mixtures. Similar considerations apply in relation to starting materials exhibiting asymetric carbon atoms as mentioned.

In the compounds of formula (I), the following significances are preferred individually or in any sub-combination:
1. Rₐ is H or CH₃;
2. R_{b} is H;
3. Ring A is unsubstituted; or is substituted by methyl in position 7;
4. Preferred heterocyclic residue as formed by NR₄R₅ is e.g. piperazin-1-y1 optionally N-substituted, e.g. by C₁₋₄alkyl, ω-hydroxy-C₁₋₄alkyl, ω-dimethylamino-C₁₋₄alkyl, C₅₋₆cycloalkyl, C₁₋₄alkyl-C₅₋₆cycloalkyl, an aromatic heterocyclic residue comprising 1 or 2 nitrogen atoms, e.g. pyridyl or pyrimidin-2-yl or 4,7-diaza-spiro [2.5] oct-7-yl; or a residue of formula β as defined above and/or optionally C-substituted, e.g. by CH₃ e.g. in positions 2, and/or 3 and/or 5 and/or 6 and/or 2,2 or 3,3 or

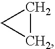

by e.g. in position 2 or 3; piperidin-1-yl optionally C-substituted, e.g. in position 4, by NH₂, —CH₂—NH₂ or piperidin-1-yl, or in position 3, e.g. by OH or NH₂; or pyrrolidinyl optionally C-substituted in position 3 by OH or NH₂;
5. Each of R₁ and R₁₁, independently, is 1-N-methyl-piperidin-4-yl; 4-methyl-piperazin-1-yl; 4-methyl-1-homopiperazinyl; 4-(2-hydroxyethyl)-piperazin-1-yl; or —X′-C_{1, 2 or 3}-alkylene-NR₇R₈ wherein X′ is a direct bond, O or NH;
6. R₁ is piperazin-1-yl optionally substituted , e.g. 1-N-methyl-piperidin-4-yl; and R₁₁ is 4,7-diaza-spiro [2.5] oct-7 yl;
7. In the residue of formula (a) either each of R₂ and R₃ is H or one of R₂ and R₃ is H and the other is F, Cl, CH₃, OCH₃ or CF₃;
8. In the residue of formula (a) either each of R₁ and R₂ is H or one of R₁ and R₂ is H and the other is F, Cl, CH₃, OCH₃ or CF₃; preferably R₂ is H and R₁ is in position 5, 6, 7 or 8, preferably in position 6;
9. In the residue of formula (b) each of R₁₂ and R₁₃ is H; or one of R₁₂ and R₁₃ is H and the other is F, Cl, CH₃, OCH₃ or CF₃; preferably R₁₃ is H and R₁₂ is in position 7;
10. In the residue of formula (b), each of R₁₂ and R₁₃ is H; R₁₁ is 4,7-diaza-spiro [2.5] oct-7 yl; or piperazin-1-yl substituted in position 3 by methyl or ethyl and optionally in position 4 by methyl.

The compounds of formula (I) are known and may be prepared as disclosed in the art, e.g. as described in U.S. Pat. No. 6,645,970, EP1490355A1, which are incorporated herein by reference. They may be prepared as disclosed or by analogy to the procedures described in these references.

Preferred compounds of formula (I) are 3-(1.H.-indol-3-yl)-4-[2-(4-methyl-piperazin-1-yl)-quinazolin-4-yl]-pyrrole-2,5-dione (referred to hereinafter as Compound A), 3-(1.H.-indol-3-yl)-4-[2-(piperazin-1-yl)-quinazolin-4-yl]-pyrrole-2,5-dione (referred to hereinafter as Compound B), 3-[3-(4,7-diaza-spiro[2.5]oct-7-yl)-isoquinolin-1-yl]-4-(7-methyl-1H-indol-3-yl)-pyrrole-2,5-dione (Compound C), in free form or in a pharmaceutically acceptable salt form, e.g. the acetate salt of 3-(1.H.-indol-3-yl)-4-[2-(4-methyl-piperazin-1-yl)-quinazolin-4-yl]-pyrrole-2,5-dione, or the acetate salt of 3-[3-(4,7-diaza-spiro[2.5]oct-7-yl)-isoquinolin-1-yl]-4-(7-methyl-1H-indol-3-yl)-pyrrole-2,5-dione.

Other PKC inhibitors to be used in accordance of the invention are compounds of formula IIa

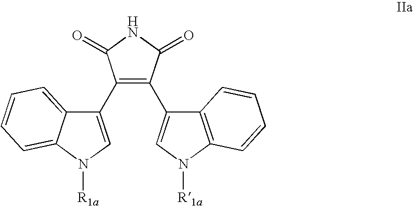

wherein
R₁ₐ is

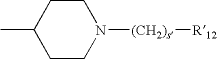

wherein either s′ is 0 and R′₁₂ is hydrogen or C₁₋₄alkyl; or s′ is 1 and R′₁₂ is pyridyl, preferably 2-pyridyl, and
R′₁ₐ is hydrogen or C₁₋₄alkyl,
or a pharmaceutically acceptable salt thereof.

The compounds of formula IIa may exist in form of hydrate or solvate.

Even more preferred are 3-(1-methyl-1H-indol-3-yl)-4-[1-{(1-pyridin-2-ylmethyl)-piperidin-4-yl}-1H-indol-3-yl]-pyrrole-2,5-dione (Compound D), or 3-(1-methyl-1H-indol-3-yl)-4-[1-(piperidin-4-yl)-1H-indol-3-yl]-pyrrole-2,5-dione (Compound E), or a pharmaceutically acceptable salt, hydrate or solvate thereof.

The compounds of formula IIa may be synthesized as known in the art, e.g. as described in U.S. Pat. No. 5,545,636.

In a series of further specific or alternative embodiments, the present invention also provides:
1. A method for treating, preventing or delaying rejection of insulin-producing cells or tissues such as islet transplantation rejection, or preventing or delaying rejection of insulin-producing tissues such as transdifferentiated hepatocytes, in particular in the case of a subject suffering from diabetes, e.g. type 1 diabetes, or pancreatitis, e.g. chronic pancreatitis, said method comprising administering to the affected subject a therapeutically effective amount of a PKC inhibitor, e.g. a compound of formula I or a compound of formula IIa, preferably compound A, B, C, D or E, or a pharmaceutically acceptable salt thereof.
2. A method for treating, preventing or delaying diabetes, e.g. type 1 diabetes, or pancreatitis, e.g. chronic pancreatitis, said method comprising administering to an affected subject a therapeutically effective amount of a PKC inhibitor, e.g. a compound of formula I or a compound of formula IIa, preferably compound A, B, C, D, or E, or a pharmaceutically acceptable salt thereof.

As herein defined, “islet transplantation” refers to islet cell autotransplantation and islet cell allotransplantation. Insulin-producing-tissues includes transdifferentiated hepatocytes as an example. These transdifferentiated hepatocytes, generated using gene activation and/or gene transfer methods, can be used for autotransplantation, allotransplantation or may be generated in situ in liver.

In another aspect the present invention provides:
3. A PKC inhibitor, e.g. a compound of formula (I) or (IIa), preferably Compound A, B, C, D or E, or a pharmaceutically acceptable salt thereof, for use in a method as defined under 1 and/or 2 above;
4. A PKC inhibitor, e.g. a compound of formula (I) or (IIa), preferably Compound A, B, C, D or E, or a pharmaceutically acceptable salt thereof, for use in the preparation of a pharmaceutical composition for use in a method as defined under 1 and/or 2 above;
5. A pharmaceutical composition for use in a method as defined under 1 and/or 2 above comprising a PKC inhibitor, e.g. a compound of formula (I) or (IIa), preferably Compound A, B, C, D or E, or a pharmaceutically acceptable salt thereof, together with one or more pharmaceutically acceptable diluents or carriers therefor.

The compounds of formula (I) may be administered in free form or in pharmaceutically acceptable salt form e.g. as indicated above. Such salts may be prepared in conventional manner and exhibit the same order of activity as the free compounds.

The compounds of formula (IIa) may be administered in free form or in form of hydrate, solvate or salt, e.g. in a pharmaceutically acceptable salt form. Such hydrates, solvates and salts may be prepared in conventional manner and exhibit the same order of activity as the free compounds.

Utility of the PKC inhibitor, e.g. in the prevention or treatment of islet transplantation rejection, as hereinabove specified, may be demonstrated in animal test methods as well as in clinic, for example in accordance with the methods hereinafter described.

A Binding affinity of PKC inhibitors to individual human PKC may be determined in an Allogeneic Mixed Lymphocyte Reaction (MLR) assay. MLR assay can be done according to known methods, e.g. mouse of human MLR assay, e.g. as disclosed in EP1337527A1, the content regarding the MLR assay being incorporated herein by reference.

B In vivo

Efficacy in the islet transplantation might be established for example as described in Nanji et al., American Journal of Transplantation 2004; 4: 526-536), the content herein being incorporated by reference.

Mice Islet Transplantation

Case 50528P1

C57BI/6 (H-2^{b}) are rendered chemically diabetic by a single intravenous injection of streptozotocin (200 mg/kg) Fully MHC-mismatched donor BALB/c islets are isolated by collagenase digestion (1 mg/ml) followed by Ficoll purification. Approximately 500 islets are transplanted under the left renal capsule of diabetic recipient mice. Allograft function is monitored by serial blood glucose measurements. Successful engraftment is defined by correction of the serum glucose level to <8 mmol/L by the third day post-transplant, and graft rejection is defined as a rise in serum glucose >15 mmol/L for 2 consecutive days. Increases of islets graft survival are obtained in animals treated with a compound of formula I administered orally at a daily dose of 1 to 30 mg/kg.

Suitable clinical studies are, e.g., randomized, double-masked, placebo-controlled clinical studies in patients with diabetes. The beneficial effects on diabetes can be determined directly through the results of these studies which are known as such to a person skilled in the art. Such studies may also be suitable to compare the effects of a monotherapy using compounds of formula I or IIa as active ingredient or a combination of such compounds with a second drug substance.

For example, 50 islet transplants with type 1 diabetes receive the test compound, e.g. a compound of formula I or IIa, or a pharmaceutically acceptable salt thereof, e.g. Compound A, B, C, D or E, at a daily dosage of e.g. 50, 200 or 400 mg or placebo administered p.o. bid. A beneficial effect is observed with the test compounds.

According to the invention, the compounds of formula (I) and (IIa) may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula (I) and (IIa) in free form or in pharmaceutically acceptable salt form in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

Preferably, the compound are administered topically, e.g. to the skin. A even more preferred form of topical administration is to the eye.

Daily dosages required in practicing the method of the present invention will vary depending upon, for example, the compound used, the host, the mode of administration, the severity of the condition to be treated. An indicated daily dosage for oral administration in the larger mammal, e.g. humans, is in the range from about 0.5 mg to about 2000 mg active ingredient, e.g. Compound A, B or C, conveniently administered, for example, in divided doses up to four times a day or in retard form.

The required dosage will of course vary depending on the mode of administration, the particular condition to be treated and the effect desired. In general, satisfactory results are indicated to be obtained systemically at daily dosages of from about 0.1 to about 100 mg/kg body weight. An indicated daily dosage in the larger mammal, e.g. humans, is in the range from about 0.5 mg to about 2000 mg, conveniently administered, for example, in divided doses up to four times a day or in retard form.

The PKC inhibitors, e.g. compounds of formula (I) or (IIa), may be administered as the sole active ingredient or together with other drugs in immunomodulating regimens or other anti-inflammatory agents, e.g. for the treatment or prevention of allo- or xenograft acute or chronic rejection or inflammatory or autoimmune disorders, or with other agents anti-diabetic drugs.

For example, the PKC inhibitors, e.g. compounds of formula (I) or (IIa), may be used in combination with cyclosporines, or ascomycines or their immunosuppressive analogs or derivatives, e.g. cyclosporin A, ISA Tx247, FK-506, ABT-281, ASM 981; an mTOR inhibitor, e.g. rapamycin, 40-O-(2-hydroxyethyl)-rapamycin, CCI779, ABT578, or a rapalog, e.g. AP23573, AP23464, AP23675, AP23841, TAFA-93, biolimus 7 or biolimus 9 etc.; corticosteroids; cyclophosphamide; azathioprene; methotrexate; an EDG receptor agonist having accelerating lymphocyte homing properties, e.g. FTY 720 or an analogue thereof; leflunomide or analogs thereof; mizoribine; mycophenolic acid or a salt thereof, e.g. sodium salt; mycophenolate mofetil; 15-deoxyspergualine or analogs thereof; a JAK3 kinase inhibitor, e.g. N-benzyl-3,4-dihydroxy-benzylidene-cyanoacetamide ω-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD 11a/CD18, CD7, CD25, CD 27, B7, CD40, CD45, CD58, CD 137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists.

For example, they may be used in combination with PPAR delta compound, for example an appropriate hypoglycemic thiazolidinedione derivative, e.g. glitazone; non-glitazone type PPARω agonists, especially N-(2-benzoylphenyl)-L-tyrosine analogues, e.g. GI-262570, and JTT501; insulin sensitivity enhancers; or AT₁ receptor antagonists, for example Diovan®, Co-Diovan® or a pharmaceutically acceptable salt thereof.
The structure of the active agents identified by generic or tradenames may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Insulin sensitivity enhancer restore impaired insulin receptor function to reduce insulin resistance and consequently enhance the insulin sensitivity.

Where the PKC inhibitors are administered in conjunction with other drugs, dosages of the co-administered compound will of course vary depending on the type of co-drug employed, on the specific drug employed, on the condition to be treated, and so forth. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

In accordance with the foregoing the present invention provides in a yet further aspect:
5. A method as defined above comprising co-administration, e.g. concomitantly or in sequence, of a therapeutically effective amount of a PKC inhibitor, e.g. a compound of formula (I) or (IIa), preferably Compound A, B, C, D or E, or a pharmaceutically acceptable salt thereof, and a second drug substance, e.g. as indicated above.

The administration of a pharmaceutical combination of the invention results in a beneficial effect, especially a synergistic effect. For example combined treatment can result in surprising prolongation of efficacy, less side-effects, lower doses of the individual drugs or improved quality of life, compared to a monotherapy. A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

With respect to the combinations according to the present invention as described hereinbefore and hereinafter they may be used for simultaneous use or sequential use in any order, e.g. for separate use or as a fixed combination.

The combinations according to the present invention comprises a “kit of parts” in the sense that both agents a and b can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration. A physician, clinician or veterinarian of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

1. A method of treating, preventing or delaying insulin-producing cell rejection comprising administering an effective amount of a PKC inhibitor of formula (I) or (IIa), 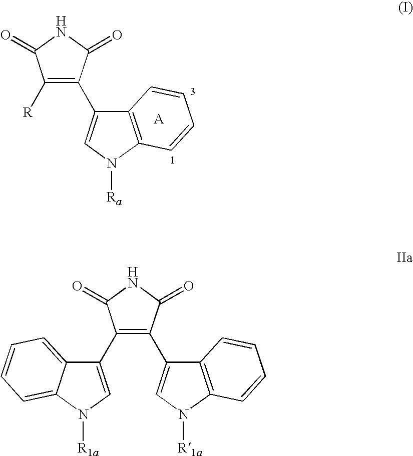
wherein
Rₐ is H; C₁₋₄alkyl; or C₁₋₄alkyl substituted by OH, NH₂, NHC₁₋₄alkyl or N(di-C₁₋₄alkyl)₂; and
R is a radical of formula (a) or (b) 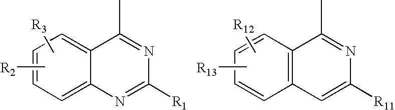
wherein
each of R₁ and R₁₁ is a heterocyclic residue; NR₄R₅ wherein R₄ and R₅ form together with the nitrogen atom to which they are bound a heterocyclic residue;
each of R₂, R₃, R₁₂ and R₁₃, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN; and
ring A is optionally substituted,
R₁ₐ is 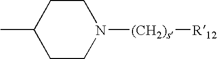
wherein either s′ is 0 and R′₁₂ is hydrogen or C₁₋₄alkyl; or s′ is 1 and R′₁₂ is pyridyl, preferably 2-pyridyl, and
R′₁ₐ is hydrogen or C₁₋₄alkyl,
or a pharmaceutically acceptable salt thereof to a subject in need of such treatment, prevention or delay.

2. The method according to claim 1 wherein R₁ is a piperazin-1-yl optionally substituted and R₁₁ is 4,7-diaza-spiro[2.5]oct-7-yl.

3. The method according to claim 1 wherein the PKC inhibitor is selected from the group consisting of 3-(1.H.-indol-3-yl)-4-[2-(4-methyl-piperazin-1-yl)-quinazolin-4-yl]-pyrrole-2,5-dione; 3-(1.H-indol-3-yl)-4-[2-(piperazin-1-yl)-quinazolin-4-yl]-pyrrole-2,5-dione; 3-[3-(4,7-diaza-spiro[2.5]oct-7-yl)-isoquinolin-1-yl]-4-(7-methyl-1H-indol-3-yl)-pyrrole-2,5-dione, and pharmaceutically acceptable salts thereof.

4. The method according to claim 3 wherein the PKC inhibitor is the acetate salt of 3-(1.H.-indol-3-yl)-4-[2-(4-methyl-piperazin-1-yl)-quinazolin-4-yl]-pyrrole-2,5-dione, or the acetate salt of 3-[3-(4,7-diaza-spiro[2.5]oct-7-yl)-isoquinolin-1-yl]-4-(7-methyl-1H-indol-3-yl)-pyrrole-2,5-dione.

5. The method according to claim 1 wherein the PKC inhibitor is selected from the group consisting of 3-(1-methyl-1H-indol-3-yl)-4-[1-{(1-pyridin-2-ylmethyl)-piperidin-4-yl)}-1H-indol-3-yl]-pyrrole-2,5-dione; 3-(1-methyl-1H-indol-3-yl)-4-[1-(piperidin-4-yl)-1H-indol-3-yl]-pyrrole-2,5-dione, and pharmaceutically acceptable salts thereof.

6. The method according to claim 4 for treating, preventing or delaying islet transplantation rejection.

7. The method according to claim 4 for treating, preventing or delaying type 1 diabetes or pancreatitis.

8. (canceled)

9. The method according to claim 5, for treating, preventing or delaying islet transplantation rejection.

10. The method according to claim 5, for treating, preventing or delaying type 1 diabetes or pancreatitis.
